(19) United States
(12) Patent Application Publication
(10) Pub. No.: US 2016/0143867 A1
(43) Pub. Date: May 26, 2016

(54) METHODS AND PHARMACEUTICAL COMPOSITIONS FOR TREATING CANCER

(71) Applicants: INSERM (INSTITUT NATIONAL DE LA SANTÉ ET DE LA RECHERCHE MÉDICALE), Paris (FR); UNIVERSITÉ PARIS DESCARTES, Paris (FR); UNIVERSITE NICE SOPHIA ANTIPOLIS, Nice (FR); CENTRE NATIONAL DE LA RECHERCHE SCIENTIFIQUE (CNRS), Paris (FR)

(72) Inventors: MARIE-PIERRE JUNIER, PARIS (FR); HERVÉ CHNEWEISS, PARIS (FR); ELIAS EL-HABR, PARIS (FR); LUIZ-GUSTAVO FEIJO-DUBOIS, PARIS (FR); THIERRY VIROLLE, NICE CEDEX 2 (FR); LAURENT TURCHI, NICE CEDEX 2 (FR); MOHAMED FAREH, NICE CEDEX 2 (FR)

(21) Appl. No.: 14/900,192

(22) PCT Filed: Jun. 20, 2014
(86) PCT No.: PCT/EP2014/063067

§ 371 Date: Dec. 21, 2015

(30) Foreign Application Priority Data
Jun. 21, 2013 (EP) 13305855.2

(51) Int. Cl. A61K 31/19; A61K 45/06

(52) U.S. Cl. CPC A61K 31/19; A61K 45/06

(57) ABSTRACT

The invention relates to methods and pharmaceutical compositions for treating cancer. In particular, the present invention relates to a compound selected from the group consisting of gamma-hydroxybutyrate (GHB), GHB derivatives, and GHB structurally-related compounds thereof, or a pharmaceutically acceptable salt thereof for use in the treatment of cancer in a subject in need thereof.

### FIELD OF THE INVENTION

The present invention relates to methods and pharmaceutical compositions for treating cancer.

### BACKGROUND OF THE INVENTION

Glioblastomas (GBM) are the most common form of primary brain tumors in adults, and the most aggressive. They resist to current therapies, and the median survival of the patients is shorter than 18 months. GBM follow the cancer stem cell (CSC) model. This concept proposes that a minority of cells within the tumor mass, with long-term self-renewal and differentiation properties, is responsible for the initiation and the growth of tumors. CSCs contribute to all the subtypes of cells that compose the tumor, including endothelial cells.

Their functional properties are associated with a molecular signature combining markers of neural and/or embryonic stem cells, and markers of mesenchymal cells. A growing body of evidences supports that these self-renewing tumor cells determine tumor's behavior, including proliferation, progression, invasion, and—most importantly—a great part of resistance to therapies. It is becoming therefore evident that failure of current treatments to eliminate glioma-initiating cells (GICs) contributes to tumor recurrence. Moreover, GICs are not restricted to adult GBM but can also be isolated from pediatric glioma of dismal outcome, such as deep infiltrating pontine glioma. Targeting GICs and their stem-like properties constitutes thus one of the main therapeutic challenges to significantly improve anti-cancer treatments. A relevant solution to target GICs is to force them to acquire a non self-renewing state. Under this non stem-like state, the cells lose their tumorigenicity and become vulnerable to therapies. It was recently demonstrated that the cluster of microRNAs miR-302-367 irreversibly commits GBM-derived GICs into a non stem-like state, and suppress their ability to initiate tumors. In addition, it was demonstrated that miR-302-367 exerts its tumor suppressor effect in a paracrine manner. The secretome of the miR-transduced cells is sufficient to trigger in vitro and in vivo the exit of GICs from their stem and tumorigenic state (WO2012010768).

### SUMMARY OF THE INVENTION

The present invention relates to methods and pharmaceutical compositions for treating cancer.

### DETAILED DESCRIPTION OF THE INVENTION

The inventors show that changes induced by miR-302-367 extend to alteration in metabolic pathways that are at the core of cell behavior. Comparison of metabolomes of miR-302-367-GICs versus control GICs, through measure of 271 metabolites by mass spectrometry, revealed significant and coordinated changes in the glutamine/glutamate metabolic pathway, as well as the synthesis pathways of neurotransmitters and neuropeptides to which it is associated, and in components of the Krebs cycle. Association of these changes with increased intra- and extra-cellular levels of 4-hydroxybutyrate (GHB), a byproduct of the GABA synthesis pathway suggests that miR-302-367 expression results in enhanced turnover/activity of this neurotransmitter metabolic pathway. Remarkably, exposure of GICs to GHB, results in a loss of clonal proliferation and self-renewal markers that mimics the paracrine effects of the miR302-367 cluster. The products of these metabolic alterations are of obvious therapeutic interest, noteworthy GHB that can cross the blood brain barrier and has been already used in human clinic. These original observations strongly suggest that metabolic alterations are instrumental in the miR-302-367 dependent irreversible repression of stem and tumor inducing properties.

Accordingly the present invention relates to a compound selected from the group consisting of gamma-hydroxybutyrate (GHB), GHB derivatives, and GHB structurally-related compounds thereof, or a pharmaceutically acceptable salt thereof for use in the treatment of cancer in a subject in need thereof.

In particular the compound of the invention is particularly suitable for inhibiting clonality, self-renewal properties and proliferation of cancer stem cells. The compound of the invention is suitable for promoting the exit of the cancer stem cells from the stem-like state. The term “cancer stem cells (CSCs)” as used herein refers to cancer cells that possess characteristics associated with normal stem cells, specifically the ability to give rise to all cell types found in a particular cancer sample. CSCs are therefore tumorigenic (tumor-forming). CSCs may generate tumors through the stem cell processes of self-renewal and differentiation into multiple cell types. In the more recent literature the initial term “cancer stem cells” has been replaced by the terms “tumor stem-like cells” or “tumor initiating cells”. Thus, the terms “tumor stem-like cells” or “tumor initiating cells” are essentially synonymous to the term “cancer stem cells”.

In some embodiments, the cancer stem cells are selected from the group consisting of liver cancer stem cells, lung cancer stem cells, brain tumor stem cells, head and neck cancer stem cells, colorectal cancer stem cells, breast cancer stem cells, leukemia cancer stem cells, pancreatic cancer stem cells, testicular cancer stem cells or gastric cancer stem cells. In a more particular embodiment the brain tumor stem cells are glioblastoma cancer stem cells or glioma cancer stem cells (i.e. glioma-initiating cells (GICs) or Glioma Stem-like Cells).

In some embodiments, the cancer stem cells are resistant to chemotherapy or radiotherapy.

Thus the compound of the present invention is particularly suitable for the treatment of liver cancer, lung cancer stem, brain tumor, head and neck cancer, colorectal cancer, breast cancer, leukemia, pancreatic cancer, testicular cancer or gastric cancer. In a particular embodiment the compound of the invention is particularly suitable for the treatment of glioma or glioblastoma. In another particular embodiment the present invention is particularly suitable for the treatment of adult or pediatric high-grade glioma or glioblastoma.

As used herein, a “GHB derivative” refers to a compound which possesses a structure similar to GHB, consisting in a carboxylic acid and an alcohol function separated by three carbon atoms, wherein at least one of the three carbon atoms may be further substituted.

As used herein, a “GHB structurally-related compound” refers to a compound which presents a common minimal structural fragment of the GHB, namely an anion as a carboxylate and a hydrogen bond acceptor donor system OH, wherein the number of carbon atoms between the carboxylate and the hydrogen bond system may vary.

As used herein, a “GHB homologue”, “GHB derivative homologue” or “GHB structurally-related compound homologue” refers to a compound which comprises a carboxylic acid and an alcohol function separated by carbon atoms as in the GHB, GHB derivative or GHB structurally-related compound structure, but wherein the number of the carbon chain is lower or higher than the three carbon atoms comprised in the GHB.

As used herein, a “GHB superior homologue” refers to a GHB homologue as defined above, wherein the number of carbon atoms between the carboxylic acid and the alcohol function is higher than three. As used herein, a “GHB inferior homologue” refers to a GHB homologue as defined above, wherein the number of carbon atoms between the carboxylic acid and the alcohol function is lower than three.

As used herein, a “GHB prodrug”, “GHB derivative prodrug” or “GHB structurally-related compound prodrug” refers to a compound which possesses a structure respectively a similar to GHB, a GHB derivative or a GHB structurally-related compound, wherein the carboxylic acid is replaced by an ester or an amide.

A “prodrug” has the capacity to be more or less rapidly hydrolysed in vivo (acid or metabolic hydrolysis), and thus produces the active species (the carboxylic moiety) after facilitation of the penetration of the lipophilic ester through the blood brain barrier.

As used herein, a “GHB bioprecursor”, a “GHB derivative bioprecursor” or “a GHB structurally-related compound bioprecursor” refers to a compound which possesses a structure respectively similar to the GHB, GHB derivative or GHB structurally-related compound, wherein the carboxylic acid function is replaced by another functional group, such as methanol, able to provide in vivo with the critical carboxylic acid group for its specific action, after oxidative metabolic transformation.

In another embodiment, the present invention relates to a GHB structurally-related compound, including an isostere, a homologue, a prodrug or a bioprecursor thereof.

Pharmaceutically acceptable salts of the compounds as defined above include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds as described above and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also encompasses the quaternization of any basic nitrogen-containing groups of the compounds disclosed herein. Water or oil-soluble or dispersible products may be obtained by such quaternization. Salt forms of the compounds as described above can be amino acid salts of carboxy groups (e.g., L-arginine, -lysine,-histidine salts).

As used herein, a “pharmaceutically acceptable counter-ion” refers to an ion which has a charge opposite to the substance to which it is associated and which is pharmaceutically acceptable.

Typically, the compound according to the invention is typically administered in a therapeutically effective amount.

By a “therapeutically effective amount” is meant a sufficient amount of a compound according to the invention to treat and/or to prevent the disease at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular patient will depend upon a variety of factors including the disease being treated and the severity of the disease; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

A further object of the invention relates to pharmaceutical compositions comprising a compound according to the invention for the prevention or treatment of cancer.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The compound according to the invention according to the invention may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The compound according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The compound according to the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In some embodiments, the compound of the invention is used in combination with a chemotherapeutic agent.

Chemotherapeutic agents include, but are not limited to, DNA alkylating agents, topoisomerase inhibitors, endoplasmic reticulum stress inducing agents, a platinum compound, an antimetabolite, vincalkaloids, taxanes, epothilones, enzyme inhibitors, receptor antagonists, therapeutic antibodies, tyrosine kinase inhibitors, boron radiosensitizers (i.e. velcade), and chemotherapeutic combination therapies.

DNA alkylating agents are well known in the art and are used to treat a variety of tumors. Non-limiting examples of DNA alkylating agents are nitrogen mustards, such as Mechlorethamine, Cyclophosphamide (Ifosfamide, Trofosfamide), Chlorambucil (Melphalan, Prednimustine), Bendamustine, Uramustine and Estramustine; nitrosoureas, such as Carmustine (BCNU), Lomustine (Semustine), Fotemustine, Nimustine, Ranimustine and Streptozocin; alkyl sulfonates, such as Busulfan (Mannosulfan, Treosulfan); Aziridines, such as Carboquone, ThioTEPA, Triaziquone, Triethylenemelamine; Hydrazines (Procarbazine); Triazenes such as Dacarbazine and Temozolomide; Altretamine and Mitobronitol.

Non-limiting examples of Topoisomerase I inhibitors include Campothecin derivatives including CPT-11 (irinotecan), SN-38, APC, NPC, campothecin, topotecan, exatecan mesylate, 9-nitrocamptothecin, 9-amino camptothecin, lurtotecan, rubitecan, silatecan, gimatecan, diflomotecan, extatecan, BN-80927, DX-8951f, and MAG-CPT as decribed in Pommier Y. (2006) Nat. Rev. Cancer 6(10): 789-802 and U.S. Patent Publication No. 200510250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11 (8): 1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2): 123-25, Crow et al. (1994) J. Med. Chem. 37(19):31913194, and Crespi et al. (1986) Biochem. Biophys. Res. Commun. 136(2):521-8. Topoisomerase II inhibitors include, but are not limited to Etoposide and Teniposide. Dual topoisomerase I and II inhibitors include, but are not limited to, Saintopin and other Naphthecenediones, DACA and other Acridine-4-Carboxamindes, Intoplicine and other

Benzopyridoindoles, TAS-I03 and other 7H-indeno[2, 1-c]Quinoline-7-ones, Pyrazoloacridine, XR 11576 and other Benzophenazines, XR 5944 and other Dimeric compounds, 7-oxo-7H-dibenz[f,ij]Isoquinolines and 7-oxo-7H-benzo[e]Perimidines, and Anthracenyl-amino Acid Conjugates as described in Denny and Baguley (2003) Curr. Top. Med. Chem. 3(3):339-353. Some agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

Examples of endoplasmic reticulum stress inducing agents include, but are not limited to, dimethyl-celecoxib (DMC), nelfinavir, celecoxib, and boron radiosensitizers (i.e. velcade (Bortezomib)).

Platinum based compound which is a subclass of DNA alkylating agents. Non-limiting examples of such agents include Carboplatin, Cisplatin, Nedaplatin, Oxaliplatin, Triplatin tetranitrate, Satraplatin, Aroplatin, Lobaplatin, and JM-216.

Non-limiting examples of antimetabolite agents include Folic acid based, i.e. dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU). Equivalents to 5-FU include prodrugs, analogs and derivative thereof such as 5′-deoxy-5-fluorouridine (doxifluroidine), 1-tetrahydrofuranyl-5-fiuorouracil (ftorafur), Capecitabine (Xeloda), S-I (MBMS-247616, consisting of tegafur and two modulators, a 5-chloro-2,4dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Examples of vincalkaloids, include, but are not limited to Vinblastine, Vincristine, Vinflunine, Vindesine and Vinorelbine.

Examples of taxanes include, but are not limited to docetaxel, Larotaxel, Ortataxel, Paclitaxel and Tesetaxel. An example of an epothilone is iabepilone.

Examples of enzyme inhibitors include, but are not limited to farnesyltransferase inhibitors (Tipifamib); CDK inhibitor (Alvocidib, Seliciclib); proteasome inhibitor (Bortezomib); phosphodiesterase inhibitor (Anagrelide; rolipram); IMP dehydrogenase inhibitor (Tiazofurine); and lipoxygenase inhibitor (Masoprocol). Examples of receptor antagonists include, but are not limited to ERA (Atrasentan); retinoid X receptor (Bexarotene); and a sex steroid (Testolactone).

Examples of therapeutic antibodies include, but are not limited to anti-HER1/EGFR (Cetuximab, Panitumumab); Anti-HER2/neu (erbB2) receptor (Trastuzumab); Anti-EpCAM (Catumaxomab, Edrecolomab) Anti-VEGF-A (Bevacizumab); Anti-CD20 (Rituximab, Tositumomab, Ibritumomab); Anti-CD52 (Alemtuzumab); and Anti-CD33 (Gemtuzumab). U.S. Pat. Nos. 5,776,427 and 7,601,355.

Examples of tyrosine kinase inhibitors include, but are not limited to inhibitors to ErbB: HER1/EGFR (Erlotinib, Gefitinib, Lapatinib, Vandetanib, Sunitinib, Neratinib); HER2/neu (Lapatinib, Neratinib); RTK class TTI: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib).

The invention will be further illustrated by the following figures and examples. However, these examples and figures should not be interpreted in any way as limiting the scope of the present invention.

FIGURES:

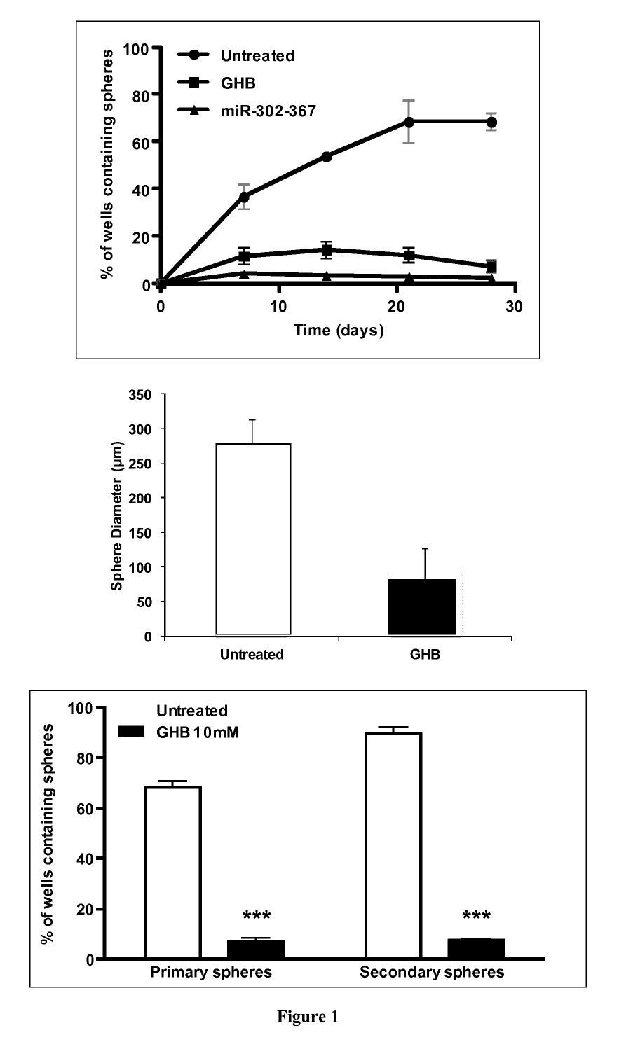

FIG. 1 shows that GHB inhibits GSC clonal and self-renewal properties.

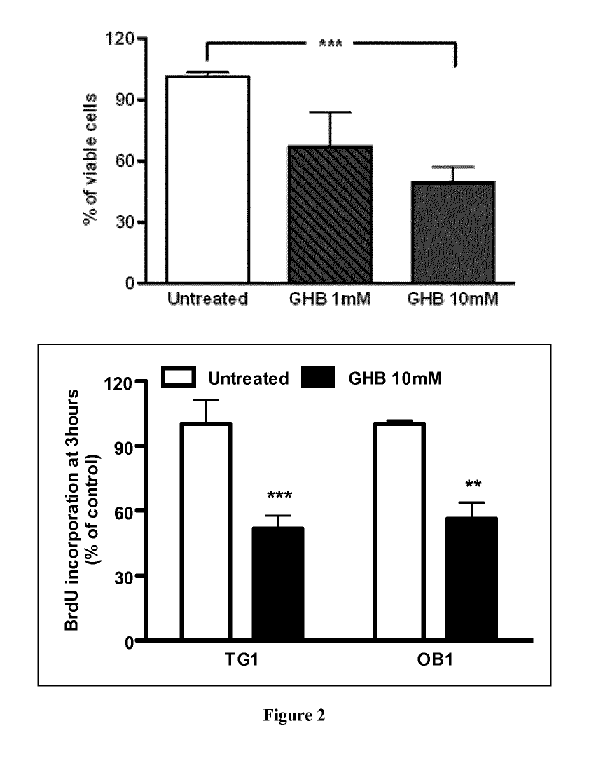

FIG. 2 shows that GHB inhibits GSC proliferation.

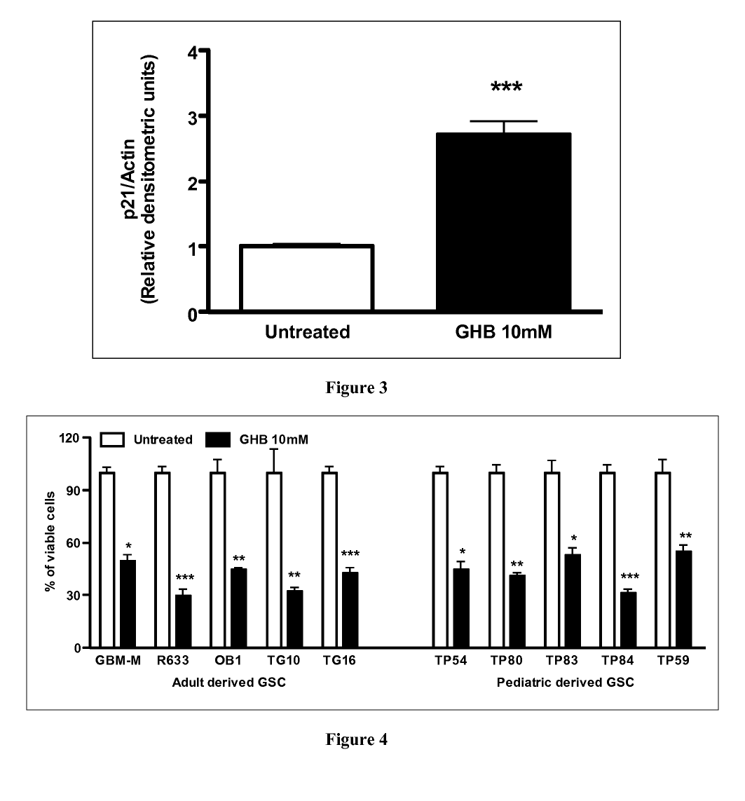

FIG. 3 shows that GHB stimulates p21/CDKN1A expression in GSC.

FIG. 4 show that GHB inhibits proliferation of GSC derived from adult and pediatric high-grade glioma.

### EXAMPLE

Irreversible repression of stem and tumor inducing properties. Since we previously observed ^{'}, as others ^{10, 13-15}, that serum exposure results in loss of stem-like and tumorigenic properties, we used this paradigm to identify miRs over or under expressed in comparison with untreated bona fide glioma-initiating cells (GICs). Functional evaluation of the miR identified as being expressed de novo in serum-treated cells demonstrated that transduction of GICs with the miR-302-367 cluster was sufficient to trigger loss of stem-like and tumor-inducing properties of GICs ¹¹. We further demonstrated that stable miR-302-367 cluster expression in self-renewing GICs (GICs-miR-302-367) promotes their irreversible exit from the stem-like state. Loss of self-renewal properties is was accompanied with repression of self-renewal markers (such as NANOG) and clonal proliferation ability. Most strikingly, the final outcome of miR-302-367 expression is the loss of the ability of GICs to initiate in vivo development of a tumor ¹¹. We have demonstrated that miR-302-367 anti-tumor effect not only involves the direct down regulation of important cell cycles regulators such as Cyclin Dl, Cyclin A, E2F1, but also the efficient and direct repression of the bad tumor prognostic marker CXCR4.

Interestingly, we found that miR-302-367 cluster was able to induce the secretion of diffusible molecules. Indeed, exposure of naïve self-renewing GICs to media conditioned by GICs stably expressing the miR-302-367 cluster, reproduced the main effects of miR-302-367 cluster, i.e. exit from sternness, loss of CXCR4, SHH, NANOG, and repression of clonal proliferation. In addition, the use of this conditioned medium strongly repressed the ability of naïve GICs to infiltrate and proliferate within organotypic cultures of cerebral tissues. These results show that factors produced under the control of miR-302-367 are able to diffuse into the microenvironment, and repress GICs properties in a paracrine manner. These results demonstrate the efficiency of miR-302-367 paracrine tumor suppressor action. This “bystander tumor suppressor effect” opens thus a unique opportunity to promote in GICs irreversible loss of stemness/tumorigenicity.

Molecular mechanisms underscoring miR-302-367 bystander effect

Delineation of the composition of the miR-302-367 secretome was first focused on small metabolite compounds.

Altered cellular metabolism in cancer cells has long been the object of attention ²¹. Their studies in epithelial cancers have noteworthy led to the identification of mutations that drive cancer pathogenesis through deregulating cellular metabolism ²², and of metabolic markers of cancer progression ²³. Despite the therapeutic potential of the use of metabolic differences between cancer and normal cells, metabolome studies in gliomas remain scarce and none has been performed on GICs.

We postulated that a change in cell phenotype as radical as that induced by miR-302-367 should result in metabolic alterations instrumental in the loss of tumorigenic and stem-like properties. We established the metabolome profile of miR302-367-GICs versus control GICs. The measure of the intracellular and secreted levels of 271 metabolites by mass spectrometry (MS), revealed noteworthy significant and coordinated changes in components of the Krebs cycle, and the glutamine/glutamate neuropeptide metabolic pathway.

Association of these changes with increased intra- and extra-cellular levels of 4-hydroxybutyrate (GHB), a byproduct of the GABA synthesis pathway ²⁴, suggests that miR-302-367 expression results in enhanced turnover/activity of this neurotransmitter metabolic pathway. This led us to test directly the effects of GHB. Exposure of GICs to GHB mimics the paracrine effects of the miR-302-367 cluster, including loss of self-renewal, down-regulation of nuclear expression of NANOG, previously demonstrated to be required for GIC self-renewal ¹¹, and inhibition of GSC clonal proliferation without affecting cell survival. We additionally showed that GHB increases the expression of the cell cycle inhibitor P21/CDKNA1, and inhibits the cell entrance into the S phase of the cell cycle. GHB is of obvious therapeutic interest, since it crosses the blood brain barrier (BBB) and has been already used in human clinic ²⁵. These original observations strongly suggest that metabolic alterations are instrumental in the irreversible repression of stem and tumor inducing properties induced by miR-302-367.

### REFERENCES

Throughout this application, various references describe the state of the art to which this invention pertains. The disclosures of these references are hereby incorporated by reference into the present disclosure.

1-7. (canceled)

8. A method for treating a cancer in a subject in need thereof, which method comprises administering, to a subject having cancer, a compound selected from the group consisting of gamma-hydroxybutyrate (GHB), GHB derivatives, and GHB structurally-related compounds thereof, or a pharmaceutically acceptable salt thereof.

9. The method according to claim 8, wherein the compound inhibits clonality, self-renewal properties and proliferation of cancer stem cells.

10. The method according to claim 9, wherein the cancer stem cells are selected from the group consisting of liver cancer stem cells, lung cancer stem cells, brain tumor stem cells, head and neck cancer stem cells, colorectal cancer stem cells, breast cancer stem cells, leukemia cancer stem cells, pancreatic cancer stem cells, testicular cancer stem cells and gastric cancer stem cells.

11. The method according to claim 10, wherein brain tumor stem cells are glioblastoma cancer stem cells or glioma cancer stem cells.

12. The method according to claim 9, wherein the cancer stem cells are resistant to chemotherapy or radiotherapy.

13. The method according to claim 8, wherein the compound is administered in combination with a chemotherapeutic agent.

14. The method according to claim 13, wherein the chemotherapeutic agent is selected from the group consisting of DNA alkylating agents, topoisomerase inhibitors, endoplasmic reticulum stress inducing agents, a platinum compound, an antimetabolite, vincalkaloids, taxanes, epothilones, enzyme inhibitors, receptor antagonists, therapeutic antibodies, tyrosine kinase inhibitors, boron radiosensitizers and chemotherapeutic combination therapies.
